(19) United States
(12) Patent Application Publication
(10) Pub. No.: US 2017/0193660 A1
(43) Pub. Date: Jul. 6, 2017

(54) Identifying a Successful Therapy for a Cancer Patient Using Image Analysis of Tissue from Similar Patients

(71) Applicant: Definiens AG, Munich (DE)

(72) Inventors: Guenter Schmidt, Munich (DE); Arno Schaepe, Starnberg (DE); Thomas Heydler, Gmund am Tegernsee (DE); Markus Rinecker, Munich (DE); Gerd Binnig, Kottgeisering (DE)

(21) Appl. No.: 15/443,672

(22) Filed: Feb. 27, 2017

Related U.S. Application Data
(63) Continuation of application No. 13/417,268, filed on Mar. 11, 2012, now abandoned.
(60) Provisional application No. 61/464,948, filed on Mar. 12, 2011.

(51) Int. Cl. G06T 7/00; A61B 6/00; G06K 9/62; G06F 19/00; G06T 7/40

(52) U.S. Cl. CPC G06T 7/0012; G06F 19/345; G06T 7/40; G06K 9/6201; A61B 6/502; A61B 6/5217; G06F 19/321; G06F 19/3443; G06T 2207/10116; G06T 2207/30068; G06T 2207/30096

(57) ABSTRACT

A clinical decision support system performs a similarity search to determine the probable outcome of applying on a current patient those clinical actions that were performed on similar patients. The system analyzes stored electronic health records of similar patients so as to recommend diagnostic and therapeutic steps for the current patient. The system receives the health record of the patient, determines which clinical actions were already applied on the patient, generates classifiers associated with potential future clinical actions, generates a success value for each health record of another patient using the classifiers, displays the health record of the other patient having the greatest success value, and indicates a proposed clinical action that is to be applied on the patient. The system also calculates a quality value indicating the probability that a sequence of clinical actions that were applied to a similar patient will be successful if applied to the patient.

### CROSS REFERENCE TO RELATED APPLICATION

This application is a continuation of, and claims priority under 35 U.S.C. §120 from, nonprovisional U.S. patent application Ser. No. 13/417,268 entitled “A Clinical Decision Support System,” filed on Mar. 11, 2012, the subject matter of which is incorporated herein by reference. Application Ser. No. 13/417,268, in turn, claims priority under 35 U.S.C. §119 from U.S. Provisional Application No. 61/464,948, entitled “A Clinical Decision Support System,” filed on Mar, 12, 2011, the subject matter of which is incorporated herein by reference.

### TECHNICAL FIELD

The present invention relates to a system for assisting a physician to arrive at a patient diagnosis, to determine the optimal sequence of clinical actions from diagnosis to therapy, and to provide hints on alternative diagnostic or therapeutic measures.

### BACKGROUND

The current procedure for a physician to use existing knowledge to determine the correct diagnosis for a patient is usually driven by personal experience, guidelines and best practices. A diagnosis frequently has a hierarchical structure, such as breast cancer, ductal carcinoma in situ, HER2 positive and ER negative. The final diagnosis for the patient's disease state is carried out in a sequence of measurements and assessments. The measurements include simple tasks, such as measuring the patient's weight and asking for her smoking habits. The measurements may also, however, be very sophisticated, such as measuring the lymph node size in computed tomography (CT) images or evaluating the HercepTest score in HER2 immunohistochemically stained tissue microscopy images. Each measurement and its assessment may be summarized as a clinical action.

The sequence of clinical actions and the decision on how to proceed may be considered as following a path in a semantic network. Each action may be considered as an edge of the network, and each decision on how to proceed and each characterization of the patient's health state can be represented as a node of the semantic network. The diagnostic procedures may be structured hierarchically with the top categories being radiology, pathology, the case history and the physical examination. On the lower level in the radiology category are X-ray and magnetic resonance tomography (MRT) results. In the pathology category are tissue examination by H&E staining and immunohistochemistry (IHC).

Therefore, finding the best sequence of clinical actions to determine the most appropriate diagnosis is equivalent to an optimization problem on how to find the shortest path in a semantic network. The starting semantic network node for the path is the current patient disease state, and the ending semantic network node is the state of the patient after treatment. Thus, the treatment options determine the sequence of steps in the diagnosis. Without available treatment options, there is no need for a diagnosis.

A method is sought for navigating from the starting semantic network node to the ending semantic network node in an optimal way.

### SUMMARY

A clinical decision support (CDS) system determines the probable outcome of applying clinical actions to a current patient by performing a similarity search that compares the health record of the current patient to the electronic health records of other patients stored in a clinical database. The CDS system includes a software application that executes on a processor of a computer. The software application analyzes the stored electronic health records of a large number of patients in order to determine those patients whose health history is most similar to that of the current patient. The software application then uses knowledge about the clinical paths followed in the past by the most similar patients and recommends potential diagnostic and therapeutic steps for the current patient.

In a first embodiment, the CDS system receives an electronic health record of the current patient that indicates a past clinical action applied to the current patient. The system performs a similarity search in a database of health records of patients in order to identify a group of patients who are similar to the current patient. The similarity search determines the similarity between two patients based on their electronic health records. Based on the electronic health records of each patient in the group of similar patients, the system calculates a corresponding quality value applicable to the current patient. Each quality value indicates the probability that a sequence of clinical actions that were applied to the corresponding similar patient will be successful if applied to the current patient. The system then indicates the clinical actions that are associated with the highest quality value. The clinical actions are indicated by displaying a representation of those clinical actions on a graphical user interface of the CDS system.

Each quality value for the current patient that corresponds to those clinical actions applied to a similar patient is determined based on estimated parameters for the current patient. For example, a quality value for the current patient can be determined based on a quality-of-life parameter for the current patient, an estimated disease free survival time for the current patient, an estimated overall survival time for the current patient or the cost of the clinical actions corresponding to the quality value.

In a second embodiment, the system receives the electronic health record of a current patient, determines that a first clinical action was already applied on the current patient, generates classifiers associated with potential future clinical actions, generates a success value for each electronic health record of another patient using the classifiers, displays the electronic health record of the other patient having the greatest success value, and indicates a proposed clinical action that is to be applied on the current patient. The system retrieves the proposed clinical action from a database in which patient medical records and associated clinical actions are stored.

In one example, the first clinical action was the acquisition of an x-ray mammography image, and the proposed clinical action is to acquire a magnetic resonance (MR) tomography image. Other examples of the proposed clinical action are: (i) a diagnosis that refines an earlier diagnosis obtained using the first clinical action, (ii) a therapy that follows a diagnosis obtained using the first clinical action, and (iii) an examination that extends the electronic health record of the current patient. At least one of the classifiers generates a success value using a fuzzy membership function to classify the stored electronic health record of another patients. The fuzzy membership function relates to an entry in the hierarchically structured electronic health record.

A representation of the proposed clinical action is then displayed on a graphical user interface of the system. The system also calculates a quality value indicating the probability that a sequence of clinical actions that were applied to a similar patient will be successful if applied to the current patient.

In a third embodiment, the system receives an electronic health record of a current patient that indicates a past clinical action applied to the current patient. The system identifies potential next clinical actions to be applied to the current patient and receives a decision as to which of the potential next clinical actions are to be applied to the current patient. The system determines a quality value for each of the potential next clinical actions that indicates the probability that each potential next clinical action will be successful if applied to the current patient. The system then determines which of the potential next clinical actions has the highest quality value and highlights on a graphical user interface a representation of the potential next clinical action having the highest quality value. The system generates a protocol that indicates the potential next clinical actions and the decision of which potential next clinical action to apply. The protocol is then displayed on a graphical user interface of the system.

In a fourth embodiment, an electronic health record of a patient is received that indicates a clinical action being applied to a current patient. A potential next clinical action to be applied to the current patient is identified. A success value for the potential next clinical action is determined that indicates the probability that the potential next clinical action will be successful if applied to the current patient. A quality value associated with a set of potential next clinical actions is determined. The quality value is based on the success value of each of the potential next clinical actions, as well as other parameters. The set of potential next clinical actions includes the potential next clinical action. The system determines that the set of potential next clinical actions has the highest quality value as compared to other sets of potential next clinical actions. The system then displays medical data on a graphical user interface supporting the determination that the set of potential next clinical actions has the highest quality value. The quality value is also calculated based on parameters such as the quality-of-life of the current patient undergoing each potential next clinical action, the estimated disease free survival time or overall survival time if the patient undergoes each potential next clinical action, and the cost of each potential next clinical action.

Other embodiments and advantages are described in the detailed description below. This summary does not purport to define the invention. The invention is defined by the claims.

### BRIEF DESCRIPTION OF THE DRAWINGS

The accompanying drawings, where like numerals indicate like components, illustrate embodiments of the invention.

FIG. 1 illustrates a semantic network with nodes that correspond to clinical actions that lead towards clinical end points.

FIG. 2 is a diagram of the structure of a novel clinical decision support system.

FIG. 3 is an exemplary screenshot of the graphical user interface of the clinical decision support system of FIG. 2.

FIG. 4 is a screenshot generated by the system of FIG. 2 showing image analysis performed on a mammogram.

FIG. 5 is a screenshot generated by the system of FIG. 2 showing the results from a similarity search performed using the image analysis of FIG. 4.

FIG. 6 is a screenshot generated by the system of FIG. 2 showing pathology information obtained from an HER2 immunohistochemically stained tissue slide.

FIG. 7 is a screenshot generated by the system of FIG. 2 showing the results from a similarity search performed using the image analysis of FIG. 6.

FIG. 8 shows the graphical user interface of FIG. 2 on which information about diagnoses and therapies has been updated.

FIG. 9 is a screenshot showing a second embodiment of the graphical user interface of the clinical decision support system of FIG. 2.

FIG. 10 is a screenshot showing a third embodiment of the graphical user interface of the clinical decision support system of FIG. 2.

FIG. 11 illustrates probability functions used to classify the shapes of stained nuclei, such as those depicted in FIG. 5.

FIG. 12 shows a screenshot used to train the clinical decision support system to classify stained nuclei, such as those depicted in FIG. 5.

### DETAILED DESCRIPTION

Reference will now be made in detail to some embodiments of the invention, examples of which are illustrated in the accompanying drawings.

A novel Clinical Decision Support (CDS) system supports a physician in arriving at a patient diagnosis. The CDS system assists the physician to figure out the optimal sequence of clinical actions from diagnosis to therapy and provides hints on alternative diagnostic and therapeutic measures. The CDS system provides help without domineering over the physician.

The novel CDS system solves the problem of how to navigate the network of clinical actions and decisions in an optimal way. The optimal path through the decision tree is determined by the patient and her preferences and by the availability and cost of clinical services. Each clinical path starts with the current patient state, which is documented in her medical health records (MHR). The path ends with the patient in her preferred state, either perfectly healthy or, if that is not achievable, with optimal quality of live or maximum life expectancy. The parameters of the clinical path optimization are comprehensive and include, for example, the patient's or the patient's health insurer's willingness to contribute to health care costs, the availability and cost of diagnostic services (e.g., PET/CT), the probability that a given diagnostic step will increase the confidence in the diagnosis, and the availability of other clinical resources (e.g., beds, doctors).

To achieve the goal of finding the optimal path for a given patient, all relevant clinical actions must be associated with a cost. In particular, a value for the clinical actions that lead to the endpoint node must be determined. By introducing a common “currency,” an optimization method is used that determines the route with the lowest overall cost when following the actions and decisions from the start point to the endpoint. Although from an ethical perspective it might be difficult to value an incremental increase in life expectancy, a pragmatic approach is to follow consensus valuations from empirical studies with an average value of [symbol] 50,000 per year of life (European Commission, CAFE 2003). By optimizing the path, the system automatically determines the optimal balance between the high cost of sophisticated diagnoses and advanced therapies with the benefits of longer life and higher quality of life.

One possible choice of an algorithm used to solve the shortest path problem is the Dijkstra algorithm. When using the Dijkstra algorithm to determine the shortest path through the semantic network, one should assume that each edge of the network is associated with a positive cost to find a path with the lowest overall cost. The CDS system uses the algorithm by associating clinical actions, such as diagnostic steps and therapies, with costs. The cost of reduced life time or quality of life is modeled using the edges leading to the endpoint node. It is important to note that in the assignment of costs to each clinical action, the costs must be risk-adjusted real costs. For example, an additional diagnosis based on magnetic resonance may have additional real costs, but due to its diagnostic power the subsequent clinical actions carry less risk, which in turn reduces the real costs.

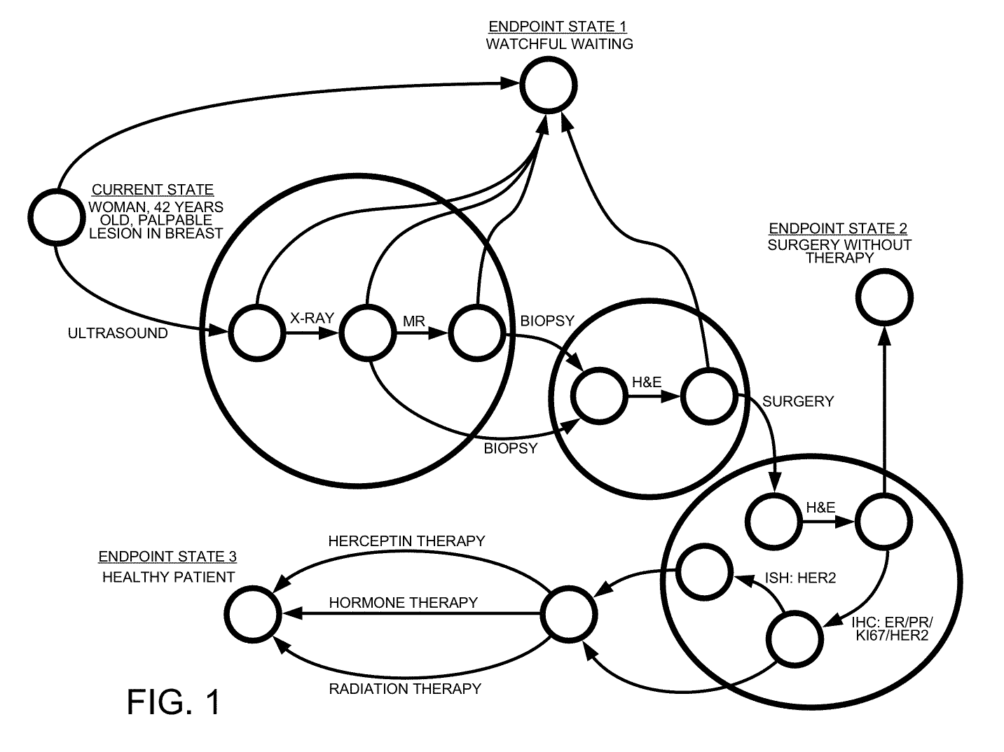

FIG. 1 shows a semantic network with nodes linked from a starting point to multiple possible end points. Each of the nodes corresponds to a clinical action that leads towards one or more clinical end points. For example, each of the actions that lead towards the “End Point State 1” results in a different cost because the additional diagnostic steps reduce the risk for the patient.

Using Knowledge from Clinical Practice

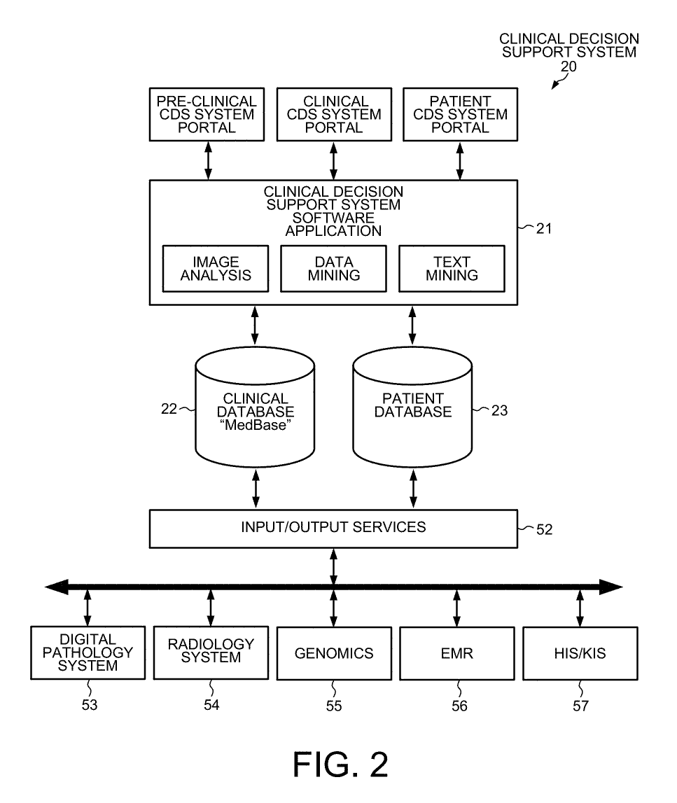

FIG. 2 is a diagram of the structure of the Clinical Decision Support System 20. The CDS system 20 includes a CDSS software application 21 that executes on a processor of a server. The CDS system uses knowledge about clinical paths generated in the past for a large number of patients, which are stored in a clinical database “MedBase” 22. For each patient in the clinical database 22, the system 20 stores the sequence of diagnostic and therapeutic steps taken for that patient in the patient database 23. These steps include all of the clinical actions (measurements, assessments and therapies), the past decisions and associated real and risk-adjusted costs.

For the patient currently being analyzed, the CDS system knows the path taken to arrive at the patient's current state. Using the patient's information, the CDS system 20 searches the clinical database 22 for patients with similar circumstances by comparing the path of the current patient with portions of the paths other patients. To determine the similarity between the path of the current patient and paths of other patients, the system 20 uses the similarity of the transited patient states (the nodes in the semantic network), the similarity of the clinical actions taken (edges in the semantic network), the similarity of the outcomes of the actions taken, and the similarity of the structures of the paths as a whole.

For example, to evaluate the similarity of the clinical action “perform a physical examination ‘edge’,” the system 20 computes the weighted Euclidean distance based on age and weight. The evaluation of a mammography image “edge” includes the computation of the similarities in the detected calcifications and masses based on the distribution patterns, densities, shapes and textures in the digital image. To obtain probabilities used in choosing clinical actions for the current patient, all similar paths from the clinical database 22 are aggregated using the similarity values as weighting factors. Using the aggregated path as an input to the algorithm for finding the optimal path provides the physician with a suggestion for the next diagnostic and therapeutic steps. Included in this suggestion is the on-demand access to the networks from which the suggestion was derived.

The value of the clinical database 22 increases with the number of patient histories it contains. Each patient history (if recorded correctly) contributes to the available network of actions and decision points. The success or failure of each diagnosis and therapy in the past enables the system 20 to repeat (or prevent) such routes. Therefore, the CDS system 20 supports a global clinical database 22 that aggregates knowledge far beyond the depth of an individual physician. To implement such a global clinical database using the constraints of privacy and ethics, all patient data contained therein should be anonymized so that each patient's identity can be retrieved only from the clinic that provided the data. For all other participating clinics, the patient's identity remains hidden.

Example 1 of Graphical User Interface of CDS System

The CDS system 20 provides a graphical user interface (GUI) for the interaction with a physician. The GUI displays patient information from the electronic health record (extracted from the hospital information system, HIS). A “findings” view of the GUI displays the patient's radiological and pathological images and other patient data (extracted from the Picture Archiving and Communication System, PACS), as well as recommendations on which clinical action should next be performed, such as additional diagnostic or therapeutics steps.

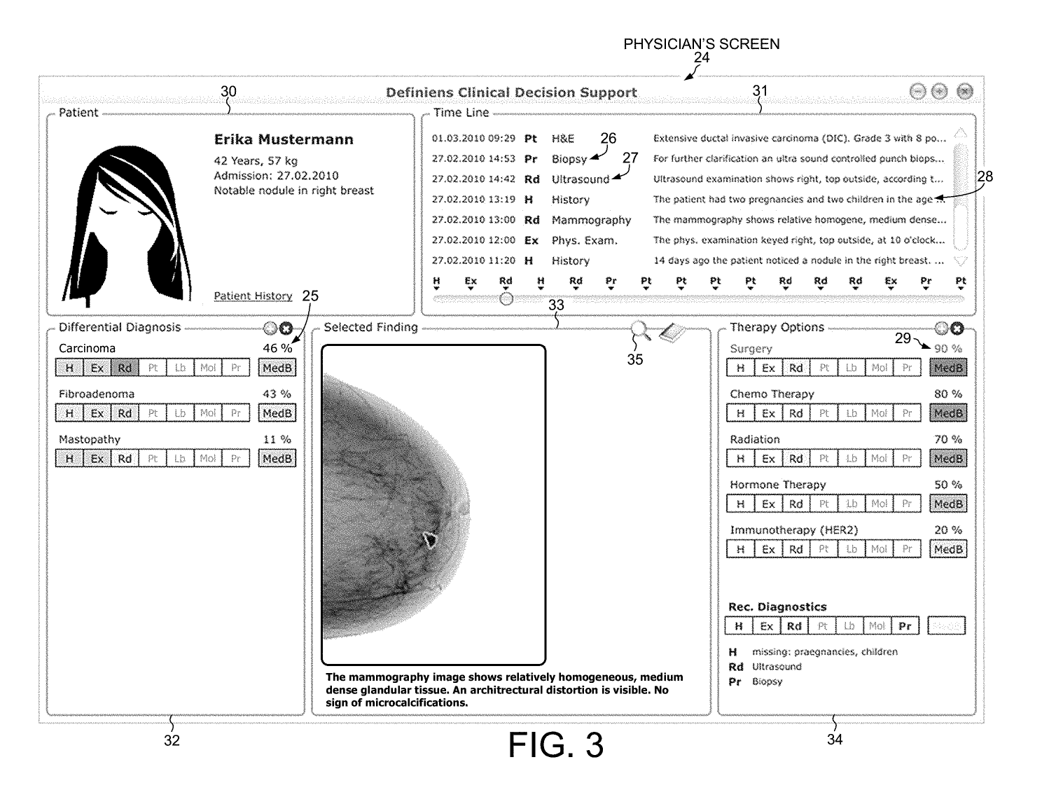

FIG. 3 is an exemplary screenshot of the “findings” view of the GUI of the CDS system 20. The sample “findings” view of the physician's screen 24 shows that the patient “Erika Mustermann” was hospitalized in the clinic with a notable nodule in her right breast. With the information about the patient's history, the physical examination, and the mammography image, the CDS system 20 concludes that the patient has a 46% probability (25) of having a breast carcinoma. Moreover, because the CDS system 20 knows the clinical guidelines for breast cancer care and has found similar cases in the clinical database “MedBase” 22, the system 20 suggests additional diagnostics, such as a biopsy (26) and an ultrasound (27), and has retrieved additional information from the patient history, such as the number of pregnancies and children (28). Based on the hypothesis that the patient has cancer, the CDS system 20 determines from the clinical database 22 that the therapy option with the highest probability (90%) of curing the cancer is surgery (29).

The GUI of FIG. 3 includes five components: a patient panel 30, a time line panel 31, a differential diagnosis panel 32, a selected finding panel 33 and a therapy options panel 34. The patient panel 30 shows the patient's photo, name, age and weight, as well as an initial diagnosis. The differential diagnosis panel 32 provides information about the potential diagnoses. Each potential diagnosis has a confidence (or classification) value that describes the likelihood that the diagnosis is correct. In addition, the steps taken to arrive at the diagnosis are highlighted in the boxes below the diagnosis. The abbreviations for the steps are: patient history (H), physical examination (Ex), radiology procedure such as mammography (Rd), pathology procedure such as biopsy (Pt), lab diagnostics such as blood test (Lb), molecular diagnostics (Mol) and clinical procedure (Pr). An example of molecular diagnostics (Mol) is immunohistochemical staining to measure HER2 protein expression status in biopsy cancer tissue. A clinical procedure (Pr) includes surgery, medication or radiation therapy.

The time line panel 31 provides information on all clinical actions performed with the current patient in chronological order. Clicking on a past point in time displays the graphical user interface as it was at the prior point in time. Displaying the past point in time allows the physician to navigate easily to previous diagnostic steps and the associated clinical data for a quick review or recap.

Two kinds of information are displayed in the therapy options panel 34 that help the physician to proceed with the patient's health care plan. First, the suggested therapy options are displayed. The therapy options correspond to the diagnosis that is selected in the differential diagnosis panel 32. Each therapy option is listed along with a success value 29 indicating the probability that the therapy option will be successful. Second, the therapy options panel 34 also includes a recommended diagnostics section in which additional clinical actions are recommended in order further to refine the current diagnosis.

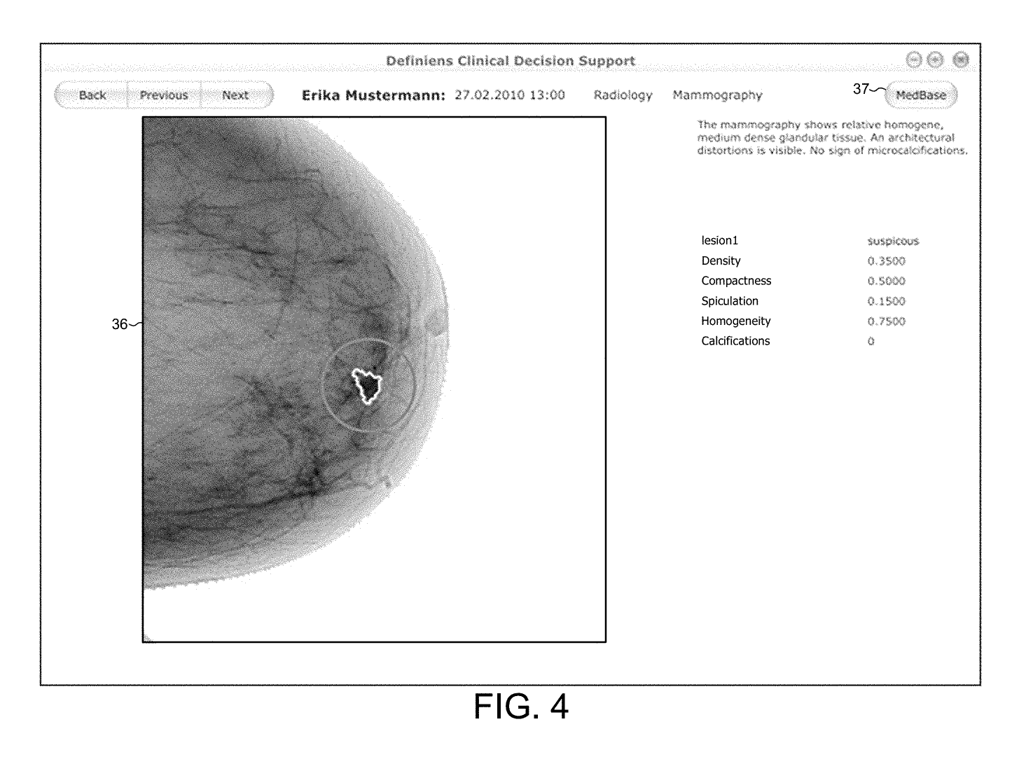

The data that drives the current clinical decision is displayed in the selected findings panel 33. This data includes information such as an x-ray mammography image, a pathology report, or a blood test result. Clicking on the magnifier symbol 35 allows the user of system 20 to navigate into the selected diagnosis in order to retrieve the underlying details. For example, when the magnifier symbol 35 of the sample GUI of FIG. 3 is selected, the additional details of the screenshot of FIG. 4 are displayed. FIG. 4 shows the image analysis of a mammogram of the patient. The CDSS software application 21 segments and classifies objects detected in the mammography digital image 36. In FIG. 4, the CDSS software application 21 has outlined a region of the digital image corresponding to a lesion. Application 21 also measures the shape, density and texture of the identified region in the digital image of the patient's breast.

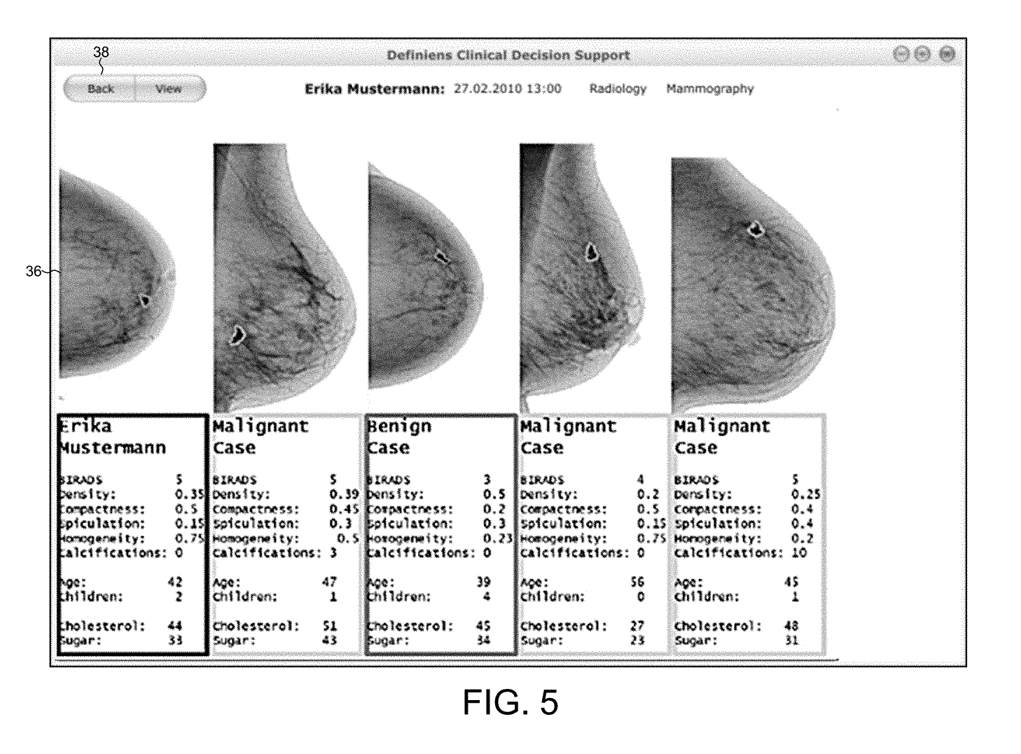

Clicking on the “MedBase” button 37 at the upper right of the screenshot of FIG. 4 opens an additional visualization of results from a similarity search in the clinical database 22. The results of the similarity search are shown in FIG. 5. At the left of FIG. 5 are the mammogram 36 and related information for the current patient. To the right of the information for the current patient are the most similar findings for four other patients stored in the clinical database 22. Because the results of the biopsies for the other four patients are known, those results can be labeled as either malignant or benign. From the similarity search, which may use lab test results as well as other clinical patient data, the CDS system 20 concludes that the patient's lesion is malignant. Clicking the “Back” button 38 returns the GUI to the screenshot of FIG. 4.

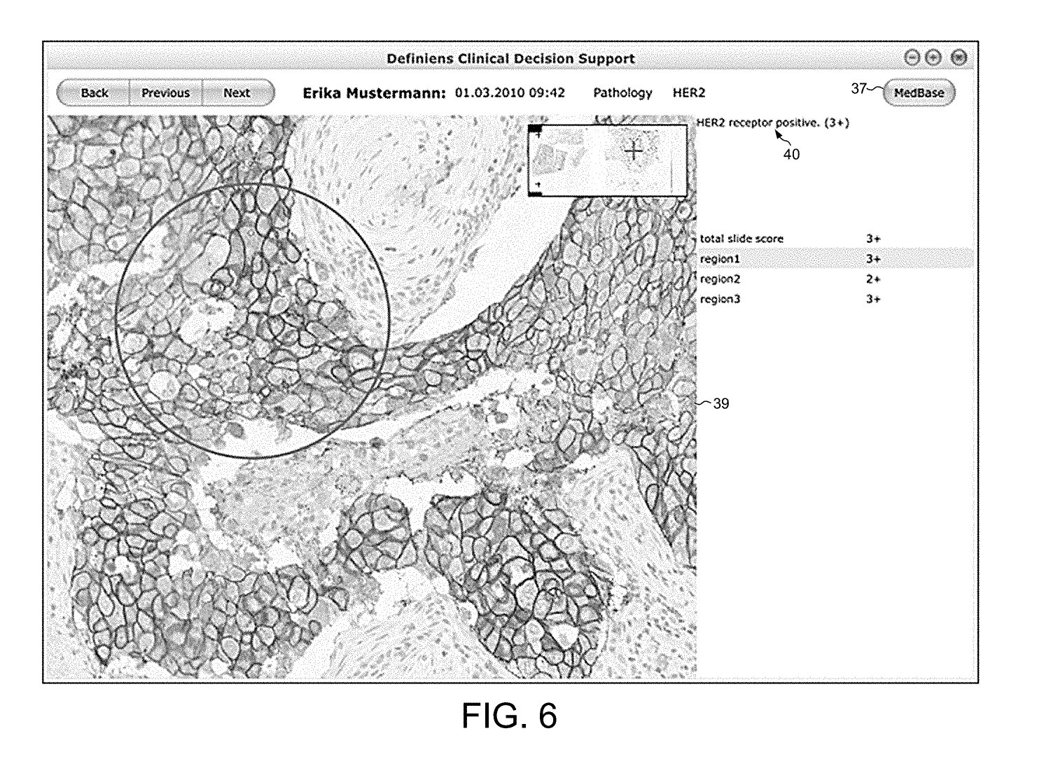

In a manner similar to the display of FIGS. 4-5, the GUI of the CDS system 20 also displays pathology information. FIG. 6 shows pathology information for the current patient in the form of an image 39 of an HER2 immunohistochemically stained tissue slide. In the example of FIG. 6, the CDSS software application 21 has performed image analysis on the image 39 of the stained tissue and has quantified the HER2 protein expression into three regions in the image. The CDSS software application 21 has determined that the overall HercepTest score for the stained tissue is 3+. The HercepTest score is displayed to the upper right 40 of the image of the stained tissue. The GUI of the CDS system 20 allows the user to navigate quickly around the entire tissue slide without leaving the application. Clicking on the “MedBase” button 37 from the screenshot of FIG. 6 again shows similar cases with their respective scores and other quantitative measurements.

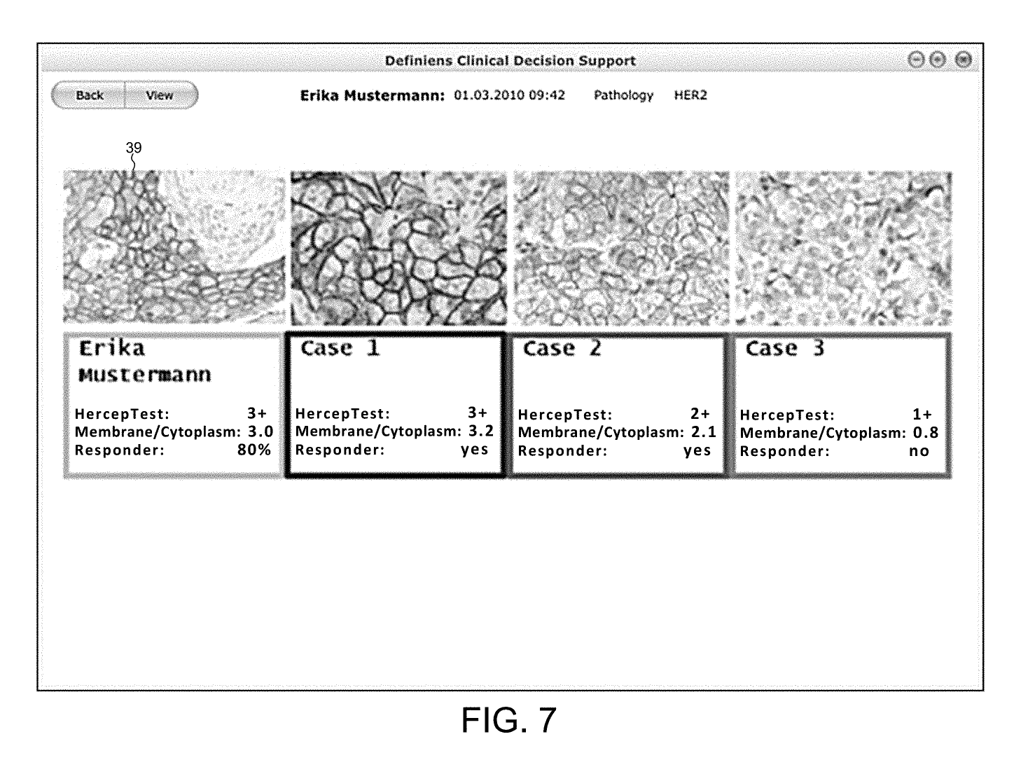

FIG. 7 shows the results of a similarity search in the clinical database 22 in which the three most similar stained tissue slides from other patients are displayed next to image 39 for the current patient. For each of the images of stained tissue, the CDSS software application 21 has performed image analysis on the image and has calculated the membrane-to-cytoplasm staining intensity ratio. The screenshot of FIG. 7 also displays the HercepTest score for each patient and whether the patient responded to adjuvant therapy. For the current patient, the CDSS software application 21 calculates a success value indicating the probability that the patient will respond to adjuvant therapy. In this case, the probability is 80%.

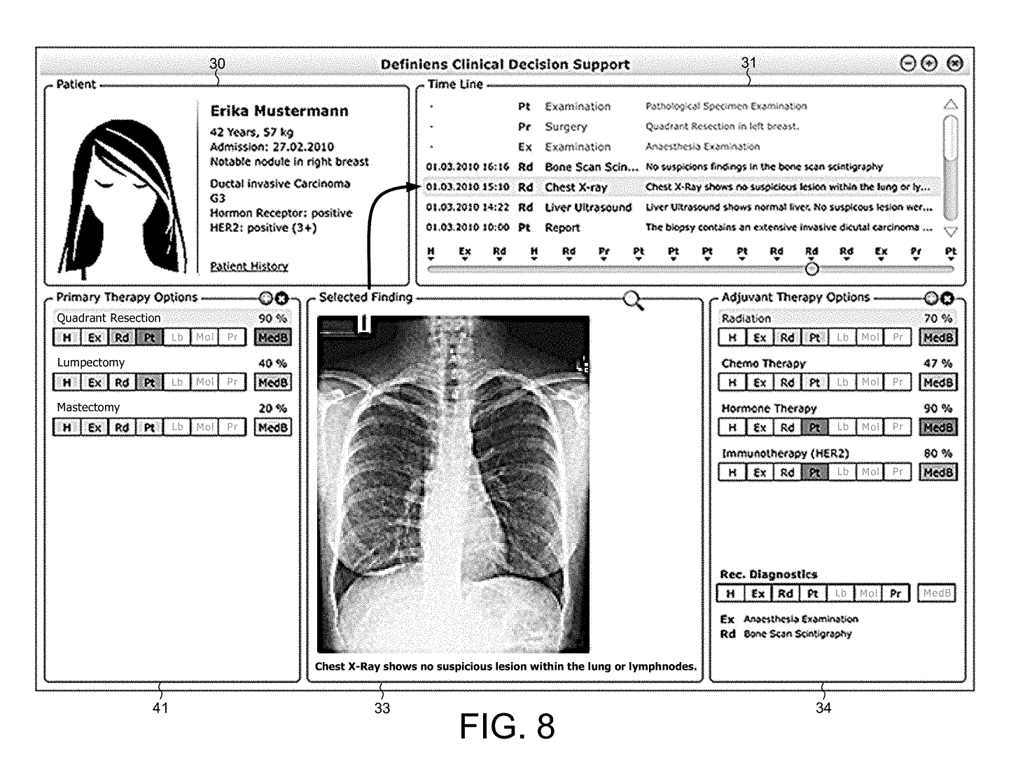

As soon as the diagnosis is sufficiently specific to start therapy, the differential diagnosis panel 32 becomes the primary therapy options panel 41, as shown in FIG. 8. The primary therapy options panel 41 lists therapy options together with an associated success value. In one embodiment, the success value indicates the probability that the therapy option will result in the desired clinical outcome. Examples of therapy options are quadrant resection, lumpectomy and mastectomy. As soon as the diagnosis is sufficiently specific to start a specific therapy, the diagnosis is displayed in the patient panel 30.

Example 2 of Graphical User Interface of CDS System

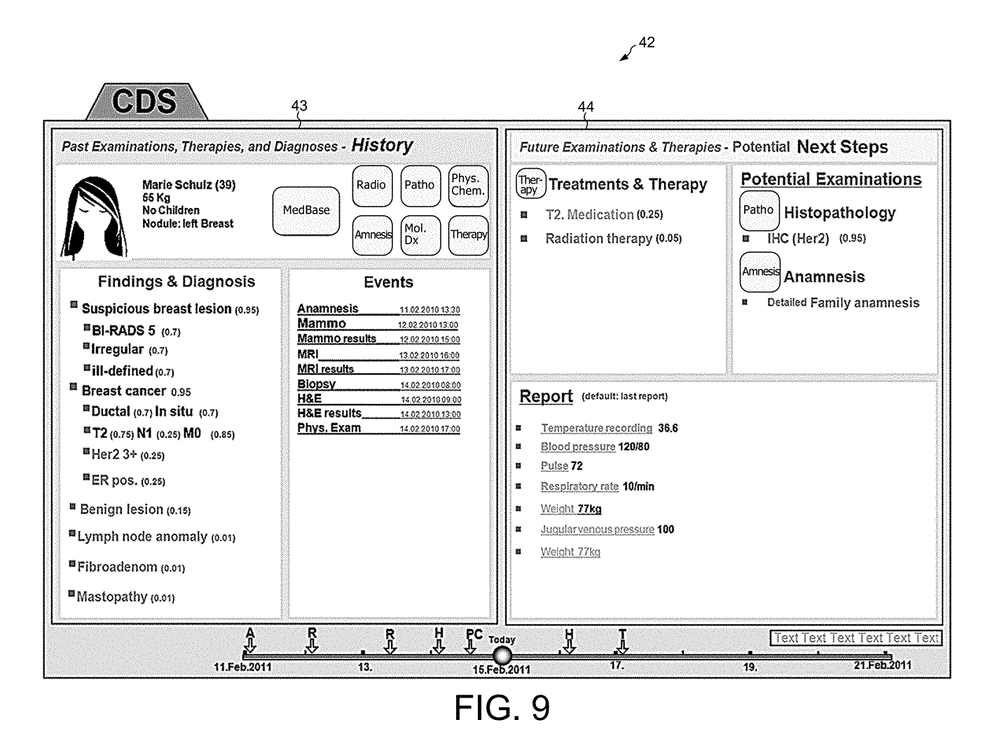

FIG. 9 shows a second version of the GUI of CDS system 20. The physician's screen 42 of the GUI is divided into two parts. The left side panel 43 relates to events that occurred in the past, whereas the right side panel 44 relates to future treatment options. The example of FIG. 9 shows information about the patient “Marie Schulz,” whose diagnosis for breast cancer has been confirmed by a physical examination, mammography images, MRI images and H&E tissue analysis by a pathologist. The dates on which the physical examination, mammography, MRI and H&E tissue analysis were performed is indicated in the “Events” section of the left side panel 43.

The CDSS software application 21 uses a diagnosis-related classifier to assign a confidence value to each diagnosis. For example, confidence value that the BI-RADS 5 diagnosis is correct is 70% (0.7), as displayed in the “Findings & Diagnosis” section of the left side panel 43. The diagnosis-related classifier is calculated using membership functions of attributes extracted from the image analysis, as well as classifier values of subordinate classifiers.

The right side panel 44 shows treatment options retrieved using a similarity search of the clinical database “MedBase” 22. The suggested clinical actions are displayed towards the upper left of the right side panel 44. A success value appears in parentheses next to each treatment or therapy indicating the probability that the clinical action will be successful if applied to the current patient. For example, the “(0.05)” next to “Radiation therapy” indicates that there is a 5% probability that radiation therapy will cure the patient's breast cancer. The right side panel 44 a includes a list of recommended potential examinations that would refine the current diagnosis.

Example 3 of Graphical User Interface of CDS System

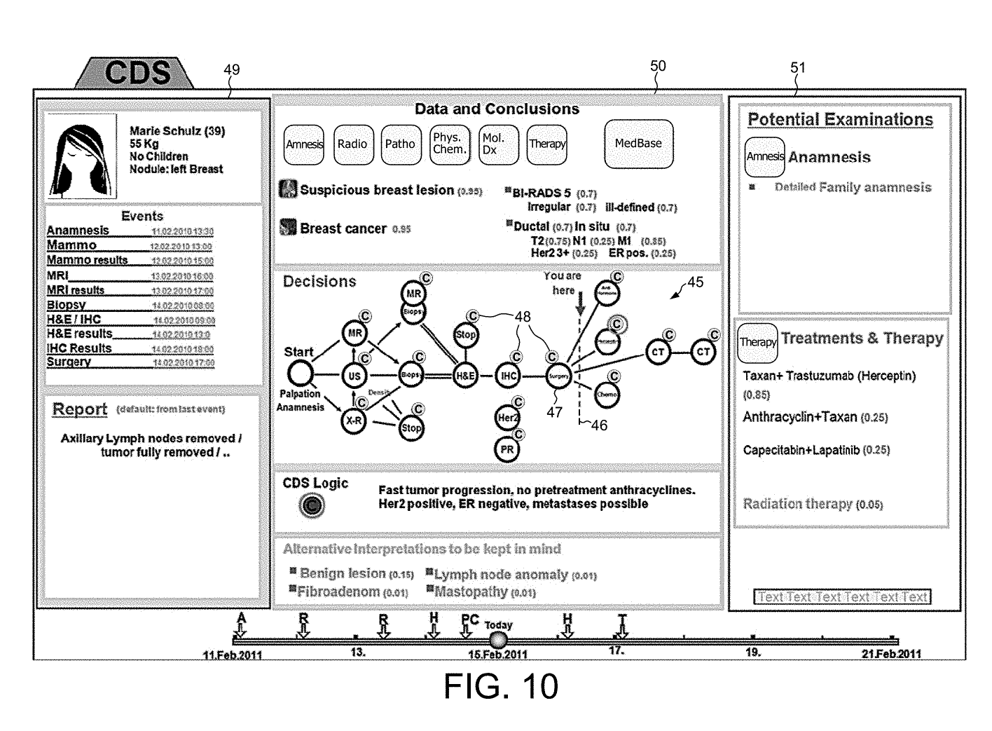

FIG. 10 illustrates a third version of the graphical user interface of the CDS system 20. At the center of the third version of the GUI is a visual representation of the network 45 of clinical actions and decision points. A dashed line labeled “You are here” 46 indicates the current point in time. The network 45 indicates that as of the current point in time, Marie Schulz's physician has three options on how to proceed after her surgery 47. Associated with each decision point is a classifier 48 labeled with a capital C. The classifier at each decision point is also called “CDS Logic.” Each classifier 48 classifies a potential next step or steps using information in the patient's electronic health record and in the clinical database “MedBase” 22. In a first embodiment of FIG. 10, the classifier generates a success value applicable to the entire path of clinical actions applied to another patient based on the electronic health record of that patient. The CDS system 20 then indicates which combination of clinical actions generates the greatest success value. In the first embodiment for example, FIG. 10 indicates that applying a therapy of Taxan and Trastuzumab (Herceptin) on the current patient “Marie Schulz” has the greatest success value (0.85).

In the GUI of FIG. 10, basic patient data is displayed in a left panel 49. This patient data includes the name and age of the patient and a short summary of the patient's diagnosis. Below the patient data is a “news ticker” that displays a short list of clinical events (actions). These actions are sorted by date. The full list of clinical actions applied in the past is accessible by the time line at the bottom. Clicking on a time line item or on a “news ticker” list entry displays the GUI of the CDS system 20 in its state as of the date of the time line item or “news ticker.”

At the top of a center panel 50 of the GUI is a set of icons that provides access to the results of different tests. Clicking on the icons reveals the results of the patient's blood tests and mammography as well as tissue-based data from pathology and gene expression data. The right-most icon of center panel 50 enables the physician to search for similar patients in the clinical database “MedBase” 22 in order to retrieve similar diagnoses, clinical actions and treatment successes. Below the icons is a set of conclusions. These conclusions are computed from the patient's clinical data and from the evaluation of the similarity search in MedBase. A conclusion is either a patient diagnosis, such as breast cancer, or the categorization of a finding in a medical image. Examples of categorizations of findings in medical images include a BI-RADS category for mammography images or an Elston-Ellis grading of H&E stained breast cancer tissue sections.

A right side panel 51 of the GUI shows information about recommended next diagnostic steps. For example, a diagnostic step could be to perform an oral “examination” in which the physician finds out more details about the patient's history. In a second embodiment of FIG. 10, the lower section of right side panel 51 displays confidence levels as opposed to success values. Each suggested treatment or therapy option is displayed together with a confidence level that indicates the probability that the option will successfully contribute to a quality measure of the patient's health care plan. A quality measure is determined based on multiple factors, such as the probability of success of the treatment or therapy option, the quality of life of the patient, the survival time, the health care costs and the patient's available health care budget. In the second embodiment, the screenshot of FIG. 10 indicates that there is an 85% chance (confidence level) that a Taxan and Trastuzumab treatment will positively contribute to the patient's health care plan.

The Clinical Decision Support System 20 has a layered architecture of data and software as shown in FIG. 2. CDS system 20 is implemented in software executing on a processor and stored on a computer-readable medium. Input/output services 52 provide access to data sources on the lowest hierarchical layer of the data network. The data sources include digital pathology repositories 53, radiology image repositories (PACS) 54, genomic gene expression databases 55, databases with electronic medical health records (EMR) 56 and other hospital information services (HIS) 57.

The patient database 23 stores data on patients currently being treated. For each patient, the database 23 includes references to the underlying data sources (e.g. PACS, HIS), information about clinical decision points and clinical actions, and the associated healthcare costs. The clinical database 22 stores data on patients whose clinical outcome and treatment success is known. Here as well, for each patient in the clinical database 22 there is a reference to the underlying data sources (e.g. PACS, HIS), information about clinical decision points and clinical actions, the actual clinical outcomes such as disease free and overall survival times, and the total health care costs actually incurred. The CDS application 21 performs various services on the data in database 22 and database 23, such as image analysis, data mining, text mining, and a combination of these functions.

The CDS system 20 includes multiple user interfaces that allow different types of users to make decisions based on the output of the system. The user interfaces provide access to data and suggestions on which clinical actions to take. For example, a user may be an employee of a pharmaceutical company that is developing and evaluating diagnostics and drugs in a pre-clinical phase. Users may also be physicians treating patients in clinics, pathologists scoring patient biopsies and resections, radiologists examining x-ray, CT, PET/CT, MRI or ultrasound images, or patients themselves seeking advice as to their best treatment.

The CDS system 20 uses classifiers to perform the analysis tasks of the system. Each classifier has several inputs that use features. A feature is a measurement result or a calculation based on another feature. A classifier creates one output value from multiple complex inputs. For example, the output value of the system is a success value or a confidence level relating to a clinical action or a sequence of clinical actions (treatments and therapies). The structure of a classifier can remain the same regardless of the specific task of the classifier.

Addressing all tasks with the same type of classifiers has the advantage that complexity is reduced. Specialization of the experts who train the classifiers or fill them manually with content is not required. The experts can more easily learn the one model and the principles that apply to all of the classifiers. While the structure of all classifiers is always alike, the contents differ, i.e. the semantic meaning and the parameters. Using a generic classifier also reduces the complexity of data mining, as only one type of algorithm must be trained. Even for very different data mining tasks, only one training and optimizing mechanism is used. A fuzzy logic classifier is well suited to represent such a generic classifier concept.

Classifiers also perform image analysis as part of the process of generating success values or confidence levels. The lowest semantic level of the data network generated by the CDS system 20 is the level of the digital images upon which image analysis is performed. The classifiers are well established and tested on this lowest semantic level of the data network. The image analysis performed by the classifiers classifies objects in the digital images through a logic or algorithmic combination of different probability functions of different features.

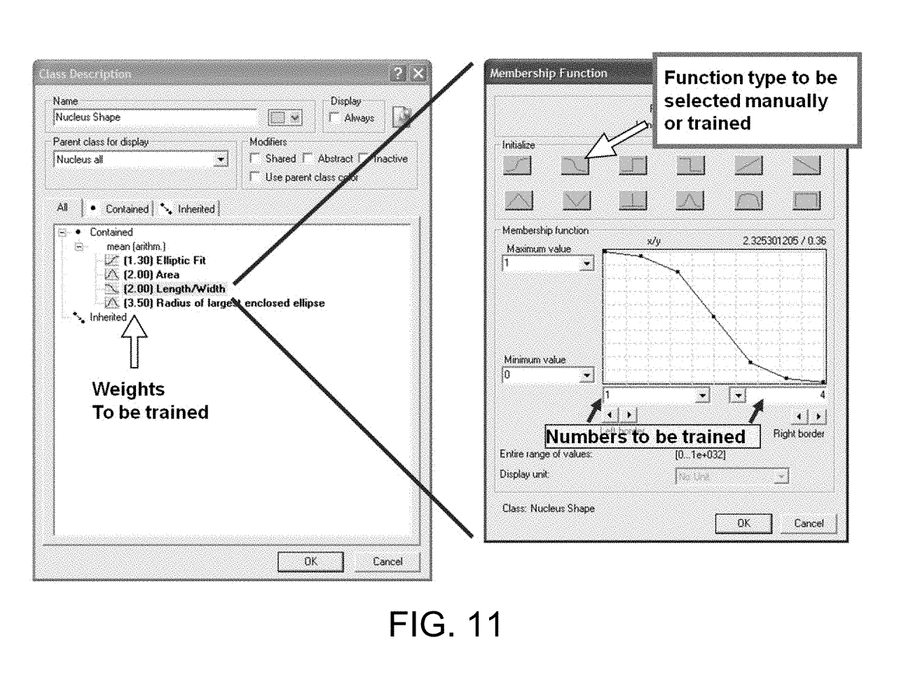

FIG. 11 shows an example of such probability functions represented by seven x/y coordinates. FIG. 11 illustrates the task of using probability functions to classify stained nuclei such as those depicted in FIG. 5 according to their shape. The shape of the stained regions of the nuclei is classified to indicate HER2 protein expression. FIG. 11 shows a classifier for a normal epithelial nucleus and demonstrates the hierarchical classification concept where contrast properties and shape properties are combined one level higher in the network to form a weighted sum.

At yet another level higher in the network, the image itself can be classified by the same principle. The image can be classified as an image with high or low quality or with respect to other criteria. In FIG. 5, the image as a whole is classified with respect to a score, in this case by calculating the probability for the HER2 3+ score in HER2 stained tissue slides. This classification stands for a variety of different score probabilities such as the Gleason score 7 or the Bloom Richardson score 5 in H&E stained images.

In one example, CDS system 20 determines a first clinical action that was applied on a patient and then uses classifiers operating on the electronic health records of the patient and other patients to determine which second clinical action should also be applied on the patient. For example, where the first clinical action is acquiring an x-ray mammography of the patient, the CDS system 20 determines that a second clinical action should be performed on the patient, such as acquiring a magnetic resonance (MR) tomography.

A classifier uses image analysis on the x-ray mammography image to determine if an MR image would provide additional information compared to the x-ray alone. In other words, the CDS system 20 determines whether a diagnosis based on the x-ray alone is reliable. If the classifier determines that the x-ray diagnosis is reliable, then the classifier could suggest options such as (i) perform no additional clinical action because the x-ray indicates a benign lesion, or (ii) proceed with a biopsy to confirm the cancer diagnosis based on the x-ray. FIG. 1 illustrates some available clinical actions in the example of a patient suspected of having breast cancer.

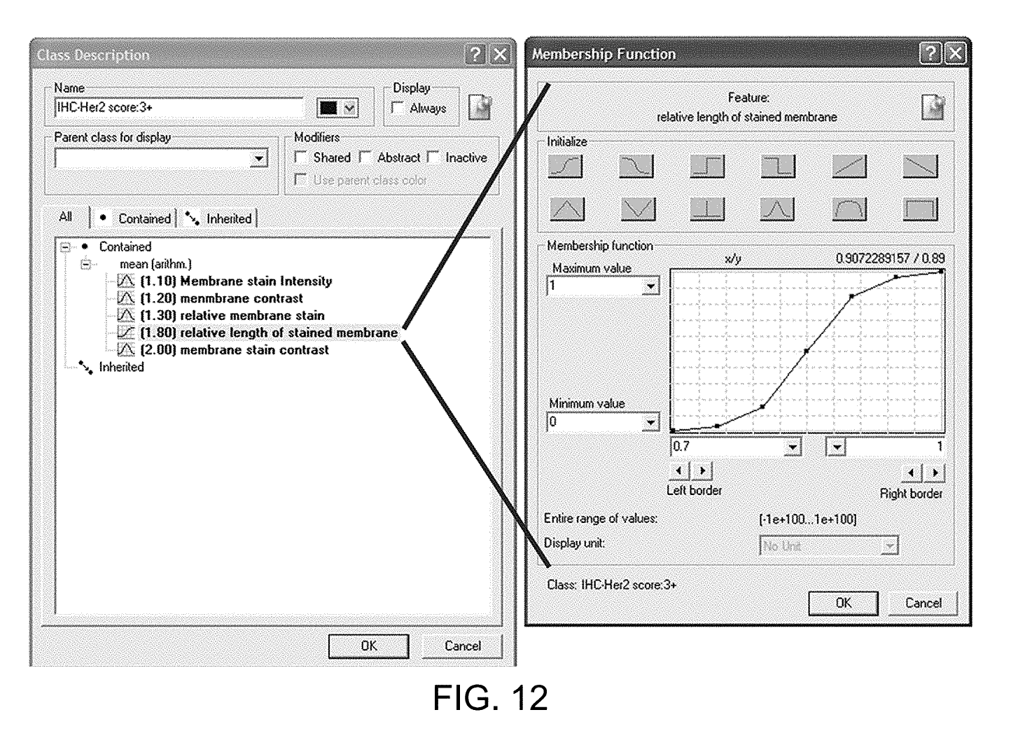

FIG. 12 shows a screenshot for training the system 20 to classify stained nuclei according to the IHC-Her2 score. FIG. 12 illustrates combining hierarchical classification for HER2 and H&E stained cells to form a new classification. Using logical expressions in a fuzzy classifier, different results from HER2 stained and H&E stained images are combined into an Herceptin score. The meaning of the Herceptin score is the probability that a given patient is a responder to Herceptin. For every confidence level proposed in CDS system 20, the classifier is trained through data mining or image analysis and is later applied to each given case so as to calculate all the different confidence levels. In order to find the most similar cases in the clinical database “MedBase” 22, the same type of classifier is used. In this case, the absolute features are replaced by the difference or the ratio of the features from the given case to the cases in the MedBase.

Where an embodiment of the CDS system 20 is implemented in software, the functions of the software may be stored on or transmitted over as one or more instructions or code on a computer-readable medium. Computer-readable media includes both computer storage media and communication media including any medium that facilitates the transfer of a computer program from one place to another. Storage media may be any available media that can be accessed by a computer. A hard disk of a server on which application 21 executes is an example of such a computer-readable medium. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer.

Although the present invention has been described in connection with certain specific embodiments for instructional purposes, the present invention is not limited thereto. Accordingly, various modifications, adaptations, and combinations of various features of the described embodiments can be practiced without departing from the scope of the invention as set forth in the claims.

1-24. (canceled)

25. A method comprising:
determining a cancer diagnosis for a cancer patient having a lesion;
determining potential therapies that could be applied to the cancer patient based on the cancer diagnosis;
measuring a characteristic of the lesion by performing image analysis on a digital image of tissue from the cancer patient;
comparing the characteristic of the lesion from the cancer patient to that characteristic of lesions detected in digital images of tissue from other patients with the cancer diagnosis and who have undergone one of the potential therapies;
identifying for each of the potential therapies a most similar patient to the cancer patient from among the other patients based on the comparing of the characteristic of the lesions of the cancer patient and the other patients;
calculating for each of the potential therapies a success value for the cancer patient based on an outcome of the potential therapy undergone by the most similar patient for that potential therapy, wherein the success value indicates a probability that each of the potential therapies will be successful if applied to the cancer patient; and
indicating on a graphical user interface the potential therapy whose success value is greatest for the cancer patient.

26. The method of claim 25, wherein the image analysis performed on the digital image of tissue from the cancer patient identifies the lesion, further comprising:
determining that the lesion of the cancer patient is malignant based on the comparing the characteristic of the lesion from the cancer patient to the characteristic of the lesions from the other patients.

27. The method of claim 25, further comprising:
determining a cancer severity score based on the image analysis on the digital image of tissue from the cancer patient; and
comparing the cancer severity score for the digital image of tissue from the cancer patient to cancer severity scores for the digital images of tissue from the other patients, wherein the identifying the most similar patient to the cancer patient for each of the potential therapies is based at least in part on the comparing of the cancer severity scores.

28. The method of claim 27, wherein the cancer severity score is taken from the group consisting of: a HercepTest score and an Elston-Ellis score.

29. The method of claim 25, wherein the image analysis measures the characteristic of the lesion in the digital image of tissue from the cancer patient, wherein the characteristic of the lesion is compared to the characteristic of the lesions in the digital images of tissue from the other patients, and wherein the characteristic is taken from the group consisting of: a shape of the lesion, a density of the lesion, a texture of the lesion, a compactness of the lesion, a spiculation of the lesion, a homogeneity of the lesion, and a membrane-to-cytoplasm staining intensity ratio of the lesion.

30. The method of claim 25, wherein the cancer diagnosis is breast cancer, and wherein the image analysis determines a number of calcifications in the digital image of tissue from the cancer patient.

31. The method of claim 25, wherein the cancer diagnosis is breast cancer, and wherein the digital image of the tissue from the cancer patient was acquired using x-ray mammography

32. The method of claim 25, wherein the cancer diagnosis is breast cancer, and wherein the tissue from the cancer patient was acquired from a tissue section of biopsy.

33. The method of claim 25, wherein the digital image upon which image analysis is performed shows tissue from the cancer patient that has been immunohistochemically stained to measure HER2 protein expression.

34. The method of claim 25, wherein the potential therapies are taken from the group consisting of: Herceptin therapy, Anthracyclin and Taxan therapy, Capecitabin and Lapatinib therapy, hormone therapy, radiation therapy, quadrant resection, lumpectomy and mastectomy.

35. A method comprising:
determining a cancer diagnosis for a cancer patient;
determining potential therapies that could be applied to the cancer patient based on the cancer diagnosis;
comparing health records of the cancer patient to the health records of other patients with the cancer diagnosis and who have undergone one of the potential therapies;
identifying a most similarly situated patient from among the other patients for each of the potential therapies;
calculating a success value for each of the potential therapies for the cancer patient based on an outcome of the potential therapy undergone by the most similarly situated patient for that potential therapy, wherein the success value indicates a probability that each of the potential therapies will be successful if applied to the cancer patient; and
indicating on a graphical user interface the potential therapy having the greatest success value for the cancer patient.

36. The method of claim 35, wherein the health records of the cancer patient include a digital image of tissue from the cancer patient, and wherein image analysis is used to compare the digital image of the tissue from the cancer patient to digital images of tissue from the other patients.

37. The method of claim 36, wherein the image analysis identifies a lesion in the digital image of tissue from the cancer patient, further comprising:
determining that the lesion of the cancer patient is malignant based on comparing the digital image of tissue from the cancer patient to digital images of tissue from the other patients.

38. The method of claim 35, wherein the cancer diagnosis for the cancer patient is breast cancer, and wherein the potential therapies are taken from the group consisting of: Taxan and Trastuzumab therapy, Anthracyclin and Taxan therapy, Capecitabin and Lapatinib therapy, hormone therapy, radiation therapy, quadrant resection, lumpectomy and mastectomy.

39. The method of claim 35, wherein the cancer diagnosis for the cancer patient is a BI-RADS 5 diagnosis.

40. The method of claim 35, wherein the success value for each of the potential therapies for the cancer patient is calculated based on an estimated disease free survival time for the cancer patient.

41. The method of claim 35, displaying on the graphical user interface a portion of the health records of the most similarly situated patient for the potential therapy having the greatest success value for the patient.

42. The method of claim 35, further comprising:
indicating on the graphical user interface a plurality of the potential therapies that in combination have the greatest overall success value for the cancer patient

43. A method comprising:
determining a first clinical action that was applied on a cancer patient;
determining potential second clinical actions that could be applied to the cancer patient after the first clinical action;
performing image analysis on a digital image of tissue from the cancer patient;
comparing the digital image of the tissue from the cancer patient to digital images of tissue from other patients who have undergone the first clinical action and one of the potential second clinical actions;
identifying a most similarly situated patient from among the other patients for each of the potential second clinical actions;
calculating a success value for each of the potential second clinical actions for the cancer patient based on an outcome of each of the potential second clinical actions undergone by the most similarly situated patient for that potential second clinical action, wherein the success value indicates a probability that each potential second clinical action will be successful if applied to the cancer patient; and
indicating on a graphical user interface the potential second clinical action whose success value is greatest for the cancer patient.

44. The method of claim 43, wherein the first clinical action is a diagnostic test the produces a diagnosis, and wherein the second clinical action is a therapy that follows the diagnosis.

45. The method of claim 43, further comprising:
determining a cancer severity score based on the image analysis on the digital image of tissue from the cancer patient; and
comparing the cancer severity score for the digital image of tissue from the cancer patient to cancer severity scores for the digital images of tissue from the other patients, wherein the identifying the most similarly situated patient to the cancer patient for each of the potential second clinical actions is based at least in part on the comparing of the cancer severity scores.

46. The method of claim 43, wherein the first clinical action is a diagnostic test the produces a diagnosis of prostate cancer, and wherein the cancer severity score is a Gleason score.

47. The method of claim 43, further comprising:
indicating on the graphical user interface a plurality of the potential second clinical actions that in combination have the greatest overall success value for the cancer patient.
